SEQUENCE LISTING (3 sequences)

SEQ ID NO: 1 - protein, 687 aa, Homo Sapien
  Soluble Flt-1 (sFlt-1) (GenBank accession number U01134)
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRGEHCNKKAVFSRISKFKSTRNDCTTQSNVKH

SEQ ID NO: 2 - protein, 221 aa, Homo Sapien
  Placental growth factor (PlGF) (GenBank accession number P49763)
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRHSPGRQSPDMPGDFRADAPSFLPPRRSLPMLFRMEWGCALTGSQSAVWPSSPVPEEIPRMHPGRNGKKQQRKPLREKMKPERCGDAVPRR

SEQ ID NO: 3 - protein, 625 aa, Homo Sapien
  Endoglin (GenBank accession number NP_000109)
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGRAAKGNCVSLLSPSPEGDPRFSFLLHFYTVPIPKTGTLSCTVALRPKTGSQDQEVHRTVFMRLNIISPDLSGCTSKGLVLPAVLGITFGAFLIGALLTAALWYIYSHTREYPRPPQ

(19) United States
(12) Patent Application Publication
(10) Pub. No.: US 2008/0071151 A1
(43) Pub. Date: Mar. 20, 2008

(54) Method and Apparatus for Diagnosing Pre-eclampsia

(76) Inventors: David C. Sogin, Highland Park, IL (US); Anton Safer, Weisenheim am Sand (DE); Brendan J. Smyth, Philadelphia, PA (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/769,705

(22) Filed: Jun. 27, 2007

Related U.S. Application Data
(60) Provisional application No. 60/818,138, filed on Jun. 30, 2006.

(51) Int. Cl. A61B 5/00

(52) U.S. Cl. 600/301

(57) ABSTRACT

A method is provided that allows a subject to be diagnosed as having one of a variety of hypertensive states, including pre-eclampsia, based on the measurement of a plurality of factors including the level of soluble fms-like tyrosine kinase 1 (sFlt-1), an obesity factor and optionally one or more additional factors, which may be physiological parameters or biomarkers. The method can be used to determine hypertensive states associated with pregnancy, or associated with anti-angiogenic drug therapy. The method is thus useful for diagnosing the hypertensive status of pregnant women, as well as patients undergoing anti-angiogenic treatment (e.g., chemotherapy).

### RELATED APPLICATION INFORMATION

This application claims priority to U.S. Application No. 60/818,138 filed Jun. 30, 2006.

### FIELD OF THE INVENTION

The present invention relates to the field of medical diagnostics and, in particular, to a method for diagnosing pre-eclampsia.

### BACKGROUND OF THE INVENTION

Hypertension is the most common medical disorder of pregnancy, with most increased maternal and fetal risk due to pre-eclampsia, a hypertensive disorder of pregnancy (HDP) unique to humans. The National Heart, Lung and Blood Institute (NHLBI) categorizes the HDP as gestational hypertension (GH), pre-eclampsia/eclampsia (PE), pre-existing chronic hypertension (CHTN) and superimposed pre-eclampsia on chronic hypertension (PE+CHTN) (see Roberts, et al. (2003) Hypertension 41(3): 437-45). It is difficult to distinguish pre-eclampsia from essential or gestational hypertension clinically, particularly in high-risk women whose pre-eclampsia is superimposed upon underlying hypertension, renal or metabolic disease. Since most of the increased risk of HDP to mother and fetus are associated with pre-eclampsia, it is important to differentiate this disorder from chronic hypertension, or gestational (non-proteinuric) hypertension. While the clinical diagnosis of pre-eclampsia is apt to be correct in over 90% of previously healthy primigravid women without underlying risk factors, only 50% of multi-gravidas with a clinical diagnosis of pre-eclampsia have this disorder in the absence of any other causes of hypertension and proteinuria. Furthermore, the clinical presentation of pre-eclampsia is itself heterogeneous, with a variable delay between the onset of hypertension and proteinuria. Pre-eclampsia can only be truly diagnosed retrospectively by the resolution of hypertension and proteinuria (generally within 26 weeks postpartum).

Considerable research has been undertaken to identify unique screening tests to detect subgroups of women at highest risk, and to distinguish pre-eclampsia from other HDP. However, the World Health Organization's (WHO) “Global Program to Conquer Pre-eclampsia” has recently assessed the usefulness of clinical, biophysical, and biochemical tests in the prediction of pre-eclampsia and concluded that there is currently no cost effective or reliable screening test for pre-eclampsia (see Conde-Agudelo, et al. (2004) Obstet Gynecol 104(6):1367-1391).

Biomarkers that have been assessed in relation to the diagnosis of PE include soluble fms-like tyrosine kinase 1 (sFlt-1; also referred to as sVEGFR1), vascular endothelial growth factor (VEGF) and placental growth factor (PlGF). The antiangiogenic sFlt-1 is a naturally occurring antagonist against circulating angiogenic VEGF and PlGF. Increased levels of sFlt-1 have been demonstrated in patients with PE (see Lam, et al. (2005) Hypertension 46:1077-1085; Maynard, et al., (2003) J. Clin Investig. 111:649-658; Chaiworapongsa, et al. (2004) Am J Obstet Gynecol 190:1541-1547; Chaiworapongsa, et al. (2005) J Maternal-Fetal and Neonatal Med 17:3-18) and circulating sFlt-1 concentrations may begin to rise in women with pre-eclampsia weeks before the onset of clinical symptoms (Levine, et al. (2004) N Engl J Med 350:672-683; Hertig, et al. (2004). Clin Chem 50(9): 1702-3; Levine, et al. (2004) N Engl J Med 350(7): 672-683; Chaiworapongsa, et al. (2005) J Matern Fetal Neonatal Med 17(1): 3-18).

Methods of predicting the occurrence of pre-eclampsia and eclampsia based on measurement of sFlt-1 have been described. For example, U.S. patent application Ser. No. 10/624,809 (Publication No. 20040126828) describes a method of diagnosing pre-eclampsia and eclampsia using sFlt-1 alone, or in combination with PlGF or VEGF. Various studies to determine the effectiveness of sFlt-1 as a diagnostic marker have also been reported (see Rodrigo, et al (2005) Am J Obstet Gynecol 193:1486-1491; Hertig, et al. (2004) Clin Chem 50:1702-1703; Levine, et al. (2006) Am J Obstet Gynecol 194:1034-1041).

Consistent with the antagonistic effect of sFlt-1, free (unbound) VEGF and free PlGF concentrations are decreased in pre-eclamptic women at disease presentation and even before the onset of clinical symptoms and, as such, VEGF and PlGF have also been assessed as potential biomarkers for the diagnosis of pre-eclampsia (see Levine, et al. (2004) N Engl J Med 350(7): 672-683). Methods of predicting the occurrence of pre-eclampsia and eclampsia based on measurement of PlGF have been described. For example, U.S. patent application Ser. No. 10/415,712 (Publication No. 20040038305) describes a method of predicting pre-eclampsia by determining the level of two or more markers selected from PlGF, plasminogen activator inhibitor-1 (PAI-1) and plasminogen activator inhibitor-2 (PAI-2). U.S. patent application Ser. No. 11/019,559 (Publication No. 20050170444) also describes a method of diagnosing pre-eclampsia using PlGF alone, or in combination with sFlt-1 or VEGF.

The ratio of sFlt1/PlGF has recently been reported as being a better predictor of pre-eclampsia (based on sensitivity and specificity) than either biomarker alone (see Levine, et al. (2004) N Engl J Med 350(7): 672-683; Buhimschi, et al. (2005) Am J Obstet Gynecol 192(3): 734-41). However, none of the above methods have been able to discriminate pre-eclampsia from other HDP.

Studies have also indicated that increased activation of angiotensin II type 1 receptors (AGTR1) may contribute to the vasoconstriction of pre-eclampsia, as circulating agonistic autoantibodies (AGTR1-AA) have been detected, even though levels of renin and angiotensin II (Ang II) are relatively low in pre-eclampsia (see Xia, et al. (2003) J Soc Gynecol Investig 10(2): 82-93; Wallukat, et al. (2003) Can J Physiol Pharmacol 81(2): 79-83; Dechend, et al. (2003) Circulation 107(12): 1632-9).

U.S. patent application Ser. No. 11/235,577 (Publication No. 20060067937) describes methods for diagnosing a pregnancy-related hypertensive disorder or a predisposition to a pregnancy-related hypertensive disorder by measuring the level or biological activity of soluble endoglin alone, or in combination with sFlt-1, VEGF or PlGF.

A “pre-eclampsia like syndrome” (PLS) is now known to occur in patients undergoing anti-angiogenic drug therapy (see Sica (2006) Clin Oncol 24(9): 1329-31). In these cases, patients are observed to have anti-angiogenic induced hypertension, which may be accompanied by proteinuria and/or other symptoms of pre-eclampsia. Hypertension and proteinuria have been observed in 25-50% of patients undergoing anti-angiogenic drug therapy, and pre-eclamptic like symptoms, including coagulopathies, neuropathies and fatigue may also occur (see Jain, et al. (2006) Nat Clin Pract Oncol 3(1): 24-40; Schöffski, et al. (2006) Ann Oncol January 17; [Epub ahead of print]; Gille, et al (2006) Exp Dermatology 15:175-186). Such pre-eclampsia like symptoms are becoming an increasing problem during anti-angiogenic therapy and there is currently no defined method to diagnose or treat such disorders.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a method and apparatus for diagnosing pre-eclampsia. In accordance with an aspect of the present invention, there is provided a method for diagnosing the hypertensive status of a subject, said method comprising the steps of: comparing a measurement of a first factor for said subject to a first pre-determined value, said first factor being a level of sFlt-1 in a sample from said subject, thereby determining the presence or absence of a first hypertensive disorder, comparing a measurement of second factor for said subject to a second pre-determined value, said second factor being a physical parameter associated with hypertensive status, thereby determining the presence or absence of a second hypertensive disorder, and diagnosing the hypertensive status of said subject based on the presence or absence of said first and second hypertensive disorders

In accordance with another aspect of the present invention, there is provided a method of evaluating whether a subject would benefit from treatment with an anti-hypertensive drug, said method comprising the steps of: comparing a measurement of a first factor for said subject to a first pre-determined value, said first factor being a level of sFlt-1 in a sample from said subject, thereby determining the presence or absence of a first hypertensive disorder, comparing a measurement of second factor for said subject to a second pre-determined value, said second factor being a physical parameter associated with hypertensive status, thereby determining the presence or absence of a second hypertensive disorder, and diagnosing the hypertensive status of said subject based on the presence or absence of said first and second hypertensive disorders, wherein the hypertensive status of said subject is indicative of whether said subject would benefit from treatment with an anti-hypertensive drug

In accordance with another aspect of the present invention, there is provided an apparatus for diagnosing the hypertensive status of a subject, said apparatus comprising: a correlation of a plurality of factors determined for each of a plurality of reference subjects having a hypertensive state with the occurrence of the hypertensive state in each of the reference subjects, said plurality of factors comprising the level of Flt-1 and a physical parameter associated with hypertension, and a means for matching an identical set of factors determined for said subject to the correlation to diagnose the hypertensive status of the subject.

In accordance with another aspect of the present invention, there is provided a method for identifying factors useful for the diagnosis of the hypertensive status of a subject, said method comprising: obtaining a data set comprising measurements of a plurality of factors associated with hypertension for each member of a reference population, said reference population comprising subjects each having a hypertensive state of normotensive or having a hypertensive disorder, and applying multivariate analysis to said data set to correlate said measurements with the hypertensive state of said subjects, thereby identifying factors useful for the diagnosis of the hypertensive status of a subject.

In accordance with another aspect of the present invention, there is provided a method of generating a functional representation of a correlation between a plurality of factors associated with hypertension with the hypertensive status of a subject, said method comprising: obtaining a data set comprising measurements of a plurality of factors associated with hypertension for each member of a reference population, said reference population comprising subjects each having a hypertensive state of normotensive or having a hypertensive disorder; applying multivariate analysis to said data set to provide a correlation between said measurements and the hypertensive state of said subjects, and generating a functional representation of said correlation.

In accordance with another aspect of the present invention, there is provided a method of diagnosing the hypertensive status of a pregnant subject as pre-eclamptic, non-preeclamptic or pre-eclamptic superimposed on chronic hypertension, said method comprising the steps of: comparing a measurement of a first factor for said pregnant subject to a first pre-determined value, said first factor being a level of sFlt-1 in a sample from said subject, thereby determining the presence or absence of pre-eclampsia, comparing a measurement of second factor for said pregnant subject to a second pre-determined value, said second factor being an indicator of obesity, thereby determining the presence or absence of chronic hypertension, and diagnosing the hypertensive status of said pregnant subject as pre-eclamptic, non-preeclamptic or pre-eclamptic superimposed on chronic hypertension based on the presence or absence of pre-eclampsia and chronic hypertension.

### BRIEF DESCRIPTION OF THE DRAWINGS

These and other features of the invention will become more apparent in the following detailed description in which reference is made to the appended drawings.

FIG. 1 presents a logic diagram representing one embodiment of the method of diagnosing pre-eclampsia of the present invention.

FIG. 2 presents a logic diagram representing additional steps in the method of diagnosing pre-eclampsia in another embodiment of the present invention.

FIG. 3 presents a decision tree in accordance with one embodiment of the invention in which three factors: body mass index (BMI), blood pressure and free sFlt-1 serum level allow pre-eclamptic patients, patients with chronic hypertension (CHTN), normotensive patients (normal) and patients with pre-eclampsia superimposed on chronic hypertension (PE+CHTN) to be distinguished.

FIG. 4 presents a scatter plot of the first principal component of free sFlt-1 (FR)+PlGF (FR_TO_PlGF) vs. PlGF for a sample of pregnant women.

FIGS. 5A-B depict the percentage variance after principal components analysis of serum parameters as described in Example 1.

FIG. 6 presents a scatter plot of the first principal component of free sFlt-1 (PCA_F1 Serum) vs. body mass index (BMI) for a sample of pregnant women; MC=Misclassification rate/sample size.

FIG. 7 presents a decision tree developed using principal components analysis that allows pre-eclamptic patients, patients with chronic hypertension (CHTN), normotensive patients (normal) and patients with pre-eclampsia superimposed on chronic hypertension (PE+CHTN) to be distinguished.

FIG. 8 presents the amino acid sequence of sFlt-1 (GenBank accession number U01134) [SEQ ID NO:1].

FIG. 9 presents the amino acid sequence of PlGF (GenBank accession number P49763) [SEQ ID NO:2].

FIG. 10 presents the amino acid sequence of endoglin (GenBank accession number NP_{—}000109) [SEQ ID NO:3].

### DETAILED DESCRIPTION OF THE INVENTION

The present invention provides for a method that allows a subject to be diagnosed as having one of a variety of hypertensive disorders, including pre-eclampsia, based on the measurement of a combination of factors including the level of soluble fms-like tyrosine kinase 1 (sFlt-1), a physiological parameter and optionally one or more additional factors, which may be physiological parameters or biomarkers. The method can be used to diagnose a hypertensive disorder associated with pregnancy, or associated with anti-angiogenic drug therapy. The method is thus useful for diagnosing the hypertensive status of pregnant women, as well as of patients undergoing anti-angiogenic treatment (e.g., chemotherapy).

Determination of the hypertensive status of a subject allows a physician to develop an appropriate treatment regimen for the subject that takes into account any risks associated with the development of a hypertensive disorder, for example seizures, cardiovascular disease and stroke.

In its simplest embodiment, the method according to the present invention provides for the diagnosis of a subject as having a hypertensive status of pre-eclampsia (PE), chronic hypertension (CHTN) or pre-eclampsia superimposed on chronic hypertension (PE+CHTN), based on the measurement of just two factors: the level of soluble fms-like tyrosine kinase 1 (sFlt-1) and an obesity factor. In another embodiment, the method of the present invention further comprises measurement of one or more additional factors and provides for the diagnosis of a subject as having a hypertensive status of PE, CHTN, PE+CHTN or normotension (i.e. normal). In a further embodiment, the method comprises measuring the level of sFlt-1 and a physiological parameter and provides for the diagnosis of a subject as having a hypertensive status of pre-eclampsia (PE) or chronic hypertension (CHTN).

The present invention further provides for an apparatus comprising a nomogram for diagnosing a subject as having one of a variety of hypertensive disorders.

As described herein, the diagnostic method of the present invention is based on the application of multivariate analysis techniques to measurements of biomarkers and physiological parameters associated with pre-eclampsia. In another aspect, the present invention thus provides for a multivariate analysis method for identifying factors useful for the diagnosis of the hypertensive status of pregnant women and/or patients undergoing anti-angiogenic treatment (e.g., chemotherapy). The multivariate analysis method can also be employed to generate a functional representation, such as a nomogram or decision tree, of the correlation between various factors that allows the diagnosis of pre-eclampsia.

### DEFINITIONS

Unless defined otherwise, all technical and scientific terms used herein have the same meaning as commonly understood by one of ordinary skill in the art to which this invention pertains.

The term “hypertensive status,” as used herein, refers to the condition of a subject with respect to the presence or absence of a hypertensive disorder, for example chronic hypertension, a hypertensive disorder associated with pregnancy (HDP), or a hypertensive disorder associated with anti-angiogenic drug therapy.

The term “pre-eclampsia,” as used herein, refers to both a multi-system disorder that is observed during pregnancy (characterized by hypertension with or before the onset of proteinuria and/or other symptoms of pre-eclampsia (see below)), as well as “pre-eclampsia-like syndrome” (PLS) associated with anti-angiogenic treatment (e.g., chemotherapy). The term “pre-eclampsia” encompasses the NHLBI HDP designation of “pre-eclampsia/eclampsia” (see below), as well the various clinical forms of the disorder, including mild, moderate, and severe pre-eclampsia. “Pre-eclampsia” also includes HELLP syndrome, a variant of severe pre-eclampsia associated with hemolysis, elevated liver enzyme levels, and low platelet count.

The term “pre-eclampsia-like syndrome (PLS)” refers to a multi-system disorder that is observed during anti-angiogenic treatment (e.g., chemotherapy), which is characterized by the new onset of hypertension with or without proteinuria, and potentially other symptoms of pre-eclampsia (see below).

The term “symptoms of pre-eclampsia” refers to both patient physical and analytical findings and complaints including hypertension (a systolic blood pressure (BP)>140 mmHg and a diastolic BP>90 mmHg after 20 weeks gestation); new onset proteinuria (1+ by dipstick on urinanalysis, >300 mg of protein in a 24 hour urine collection, or random urine protein/creatinine ratio >0.3), and resolution of hypertension and proteinuria by 26 weeks postpartum, or upon cessation of anti-angiogenic therapy. The symptoms of pre-eclampsia can also include renal dysfunction, glomerular endotheliosis, edema, neuropathy, coagulopathy and/or fatigue.

A “hypertensive disorder of pregnancy (HDP)” is used herein in the context defined by the National Heart, Lung and Blood Institute (NHLBI) (see Roberts, et al. (2003) Hypertension 41(3): 437-45). The NHLBI classify the HDP into 4 categories:
Pre-eclampsia (PE) defined as: blood pressure (BP)≧140/90; >300 mg/24 h proteinuria at >20 weeks gestation.
Chronic Hypertension (CHTN) defined as: BP≧140/90 prior to pregnancy or <20 weeks gestation.
Superimposed pre-eclampsia on chronic hypertension (PE+CHTN) defined as: the development of newly increased proteinuria in a woman with existing chronic hypertension >20 weeks of gestation.
Gestational Hypertension (GH) defined as: hypertension without proteinuria at >20 weeks.

In the context of the present invention, the term “factor” refers to a measurable physiological parameter or biomarker that is associated with the hypertensive status of a subject. Examples include, but are not limited to, physiological parameters (biosignals) such as indicators of obesity (for example, weight, body mass index (BMI), amount of body fat, waist-to-hip ratio, and the like), blood pressure (systolic and diastolic), pulse pressure, mean arterial pressure, cardiac output, aortic stiffness, microvascular resistance, systemic vascular resistance (SVR), heart rate variability (HRV) and heart rate turbulence (HRT); and various biomarkers such as soluble Flt-1 (sFlt-1), placental growth factor (PlGF), soluble endoglin (sENG), vascular endothelial growth factor (VEGF), activin, angiotensin II (ang II), angiotensin II type 1 receptor agonistic autoantibodies (AGTR1-AA), interleukins (for example IL-6 and IL18) and indicators of oxidative stress (for example glutathione peroxidase (GPX), superoxide dismutase (SOD), and malondialdehyde (MDA)).

The term “body mass index” or “BMI,” as used herein, refers to a measure of the weight of a person scaled according to height. The index can be calculated from a subject's weight and height using the equation:

$$BMI = \frac{Weight ( kg )}{Height squared ( {m}^{2} )}$$

Alternatively, BMI can be calculated using Imperial units using the equation:

$$BMI = 703 \frac{Weight ( lb )}{Height squared ( {in}^{2} )}$$

The term “multivariate analysis,” as used herein, refers to a procedure that involves observation and analysis of more than one statistical variable at a time and includes models such as canonical correlation analysis, regression analysis, principal component analysis (PCA), discriminant function (or canonical variate) analysis (DFA), multidimensional scaling, linear discriminant scaling, logistic regression, multivariate analysis of variance (MANOVA) and artificial neural networks, as well as various combinations of these models as are known in the art.

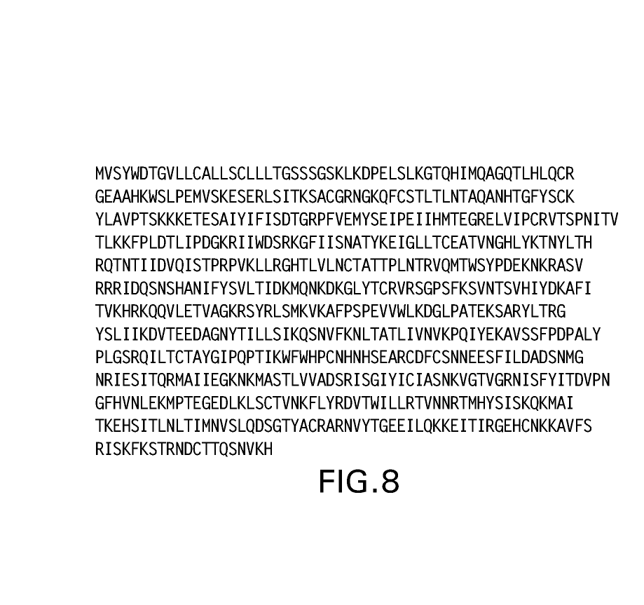

The term “soluble Flt-1 (sFlt-1),” as used herein, refers to the soluble form of the Flt-1 receptor (also known as sVEGF-R1), that is substantially identical to the protein defined by GenBank accession number U01134 [SEQ ID NO:1] (FIG. 8), and that has sFlt-1 biological activity. The biological activity of an sFlt-1 polypeptide can be assayed using various standard method, for example, by assaying sFlt-1 binding to VEGF or PlGF. As used herein, sFlt-1 includes any sFlt-1 family member or isoform. sFlt-1 can also mean degradation products or fragments that result from enzymatic cleavage of the Flt-1 receptor and that maintain sFlt-1 biological activity.

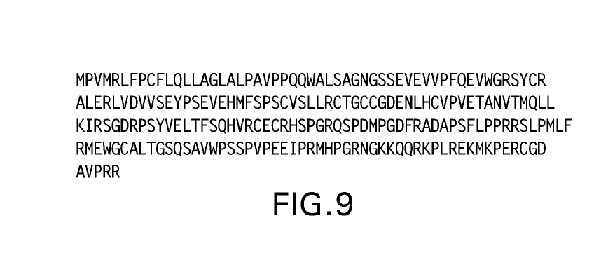

The term “placental growth factor (PlGF),” as used herein, refers to a mammalian growth factor that is substantially identical to the protein defined by GenBank accession number P49763 [SEQ ID NO:2] (FIG. 9) and that has PlGF biological activity. PlGF is a glycosylated homodimer belonging to the VEGF family and can be found in two distinct isoforms through alternative splicing mechanisms (PlGF-I and PlGF-II), both of which are encompassed by the term “PlGF” as used herein.

The term “vascular endothelial growth factor (VEGF),” as used herein, refers to a mammalian growth factor that is substantially identical to one of the known isoforms of VEGF (VEGF-A, VEGF-B, VEGF-C, VEGF-D, VEGF-E, VEGF189, VEGF165, or VEGF 121), and has VEGF biological activity. The biological activity of native VEGF includes the promotion of selective growth of vascular endothelial cells or umbilical vein endothelial cells and induction of angiogenesis.

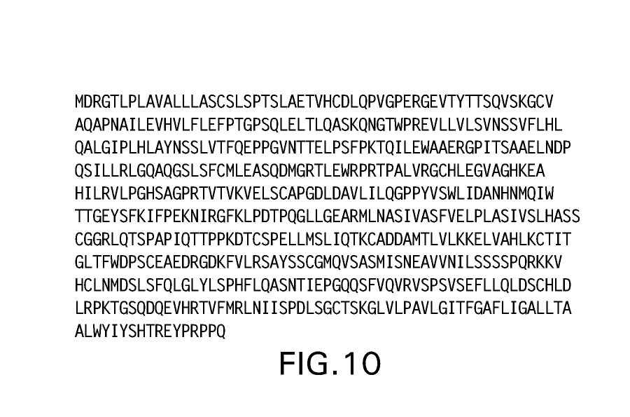

The term “endoglin,” as used herein, refers to a mammalian growth factor that is substantially identical to the protein defined by GenBank accession number NP_{—}000109 [SEQ ID NO:3] (FIG. 10), P17813 or CAA50891 (also known as CD105) that has endoglin biological activity. Endoglin can be found in one of two distinct isoforms, L and S, which differ in their cytoplasmic tails by 47 amino acids. Both isoforms are encompassed by the term endoglin as used herein. Endoglin biological activities, which can be assayed by art known methods include binding to TGF-beta family members such as activin-A, BMP 2, BMP-7, TGF-beta1 and TGF-beta3; induction of angiogenesis, regulation of cell proliferation, attachment, migration, invasion; and activation of endothelial cells. “Soluble endoglin” (sENG) refers to a circulating, non-membrane bound form of endoglin, which includes at least a part of the extracellular portion of the protein, for example, the portion including amino acids 1-437. Soluble endoglin can also include circulating degradation products or fragments that result from enzymatic cleavage of endoglin and that maintain endoglin biological activity.

The term “substantially identical,” as used herein in relation to an amino acid sequence indicates that, when optimally aligned, for example using the methods described below, the amino acid sequence shares at least 70%, 75%, 80%, 85%, 90%, 95%, 96%, 97%, 98%, 99%, or 100% sequence identity with a defined second amino acid sequence (or “reference sequence”). “Substantial identity” can refer to various types and lengths of sequence, such as full-length sequence, biologically active fragments, or functional domains. Percent identity between two polypeptides can be determined by various methods known in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed pp 353-358; BLAST program (Basic Local Alignment Search Tool; (Altschul, S. F., W. Gish, et al. (1990) J Mol Biol 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can readily determine appropriate parameters for measuring alignment, including algorithms needed to achieve maximal alignment over the length of the sequences being compared. In general, for proteins, the length of comparison sequences will be at least 10 amino acids. One skilled in the art will understand that the actual length will depend on the overall length of the sequences being compared and may be at least 20, 30, 40, 50, 60, 70, 80, 90, 100, 110, 120, 130, 140, 150, 200, 250, 300, 350, or 400 amino acids, or it may be the full-length of the amino acid sequence.

The terms “subject” and “patient,” as used interchangeably herein, refer to an individual for whom hypertensive status is to be determined.

As used herein, the term “about” refers to a +/−10% variation from the nominal value. It is to be understood that such a variation is always included in any given value provided herein, whether or not it is specifically referred to.

Methods of Diagnosis

The method of the present invention provides for the diagnosis of the hypertensive status of a subject by measurement of a combination of at least one biomarker and at least one physiological parameter. The method generally comprises a minimum of measuring a level of sFlt-1 in a sample of blood from the subject and measuring a physiological parameter for the subject. Comparison of these measurements with pre-determined values allows the hypertensive status of the subject to be determined, for example, to distinguish between pre-eclampsia and chronic hypertension.

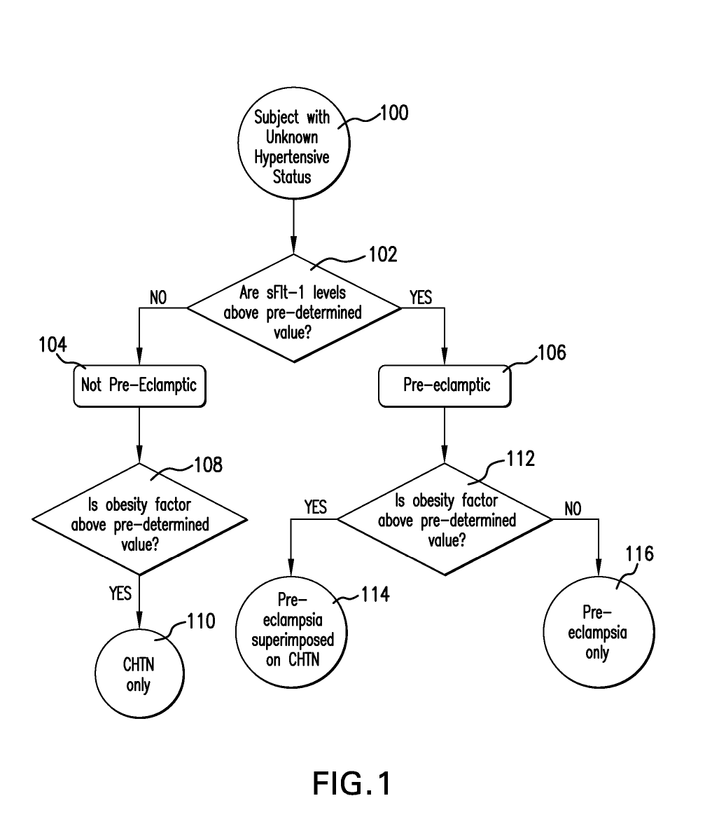

In the simplest embodiment of the present invention, the method comprises the above-described steps and provides for the diagnosis of the hypertensive status of a subject as being pre-eclamptic (PE), as having chronic hypertension (CHTN) or having pre-eclampsia superimposed on chronic hypertension (PE+CHTN). This embodiment of the invention is outlined in the flow diagram provided in FIG. 1. Thus, for a subject with unknown hypertensive status (100), the level of sFlt-1 is determined and compared to a pre-determined value. At 102 if the level of sFlt-1 is below the pre-determined value, then the subject is classified as not having pre-eclampsia (104), whereas if the level of sFlt-1 is above the pre-determined value the subject is classified as having pre-eclampsia (106). If the subject is classified as not having pre-eclampsia (104), and the obesity factor is above the pre-determined value at 108, then the subject is classified as having chronic hypertension (110). If the subject is classified as having pre-eclampsia (106), and the obesity factor is above the pre-determined value at 112, then the subject is classified as having pre-eclampsia superimposed on chronic hypertension (114), whereas if the obesity factor is below the pre-determined value at 112, then the subject is classified as having pre-eclampsia only (116).

In other embodiments, the method comprises measurement of one or more other factors and allows the hypertensive status of subjects to be defined further. In various embodiments, the one or more additional factors are blood pressure, vascular resistance, heart rate variability, level of PlGF, level of soluble endoglin, or a combination thereof.

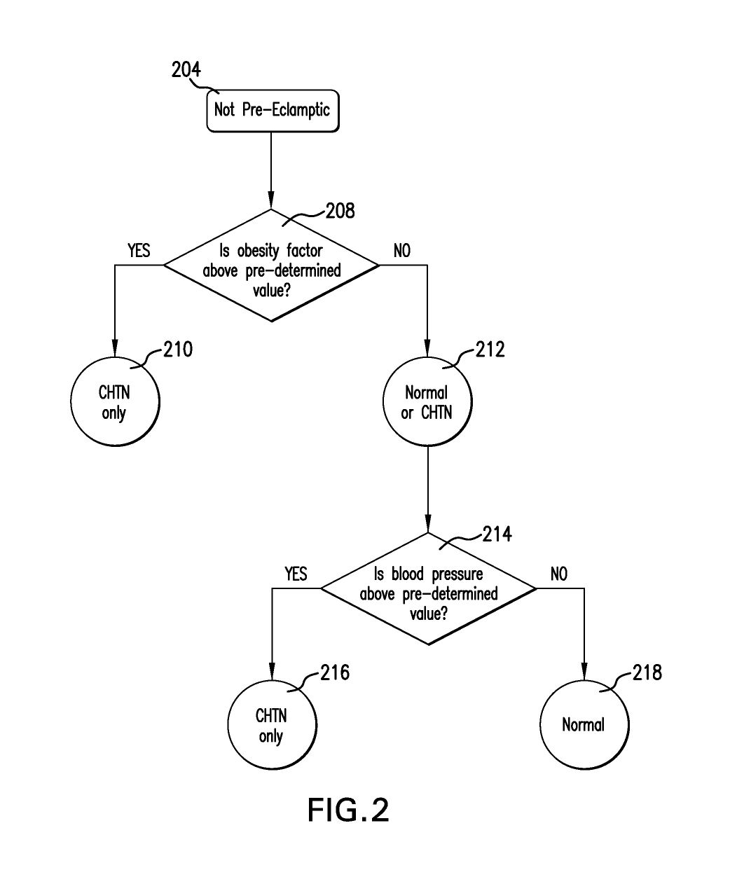

Thus, in another embodiment of the present invention, the method comprises the additional steps outlined in the logic diagram provided in FIG. 2 of measuring the blood pressure of a subject who has been diagnosed as not having pre-eclampsia (for example, by following the method outlined in FIG. 1), and comparing the blood pressure measurement obtained with a pre-determined value thus determining whether the subject has existing chronic hypertension. Referring to FIG. 2, for a subject who has been classified as not having pre-eclampsia (204), the obesity factor is measured and compared to a pre-determined value. If the measured obesity factor is above the pre-determined value at 208, then the subject is classified as having chronic hypertension (CHTN) (210), whereas if the measured obesity factor is below the pre-determined value at 208, then the subject may be normal or CHTN (212). For a subject who may be normal or CHTN (212), supine blood pressure is measured and compared to a pre-determined value. If the measured blood pressure is higher than the pre-determined value at 214, then the subject is classified as CHTN (216), whereas if the measured blood pressure is lower than the pre-determined value at 214, then the subject is classified as normal (218).

In a further embodiment of the present invention, the method comprises measuring a level of sFlt-1 in a sample of blood from the subject and measuring blood pressure for the subject and comparing these measurements with pre-determined values to provide for the diagnosis of the hypertensive status of the subject as being pre-eclamptic (PE) or as having chronic hypertension (CHTN). In another embodiment, the blood pressure measurement is systolic blood pressure. The method can further comprise measurement of one or more other biomarkers or physiological parameters.

Factors

For the purposes of the present invention, a factor is a measurable physiological parameter or biomarker that is associated with the hypertensive status of a subject. A number of measurable physiological parameters and biomarkers are known in the art to be related to the presence of hypertension and/or pre-eclampsia and are, therefore, suitable for incorporation into the method of the present invention.

Physiological Parameters

Suitable physiological parameters (biosignals) that can be measured in accordance with the method of the present invention include, but are not limited to, various indicators of obesity, as well as other physiological parameters that can be measured non-invasively including temperature, blood pressure (systolic and diastolic), pulse pressure, mean arterial pressure, pulse oximetry, cardiac output, aortic stiffness, microvascular resistance, systemic vascular resistance (SVR), electrocardiography (ECG), heart rate variability (HRV), heart rate turbulence (HRT) and ejection fraction, and physiological parameters that involve invasive measurements, such as pulmonary central wedge pressure (PCWP), cardiac index (CI).

In one embodiment of the present invention, the physiological parameters measured in the method are parameters that can be measured non-invasively. In another embodiment, the method of the present invention includes the measurement of an indicator of obesity. Various physiological factors are known in the art to be measurable indicators of obesity. Examples include, but are not limited to, weight, body mass index (BMI), waist-to-hip ratio, waist circumference, conicity index, abdominal height (or sagittal diameter), and the use of underwater weighing tanks or the skin-fold (caliper or “pinch”) test to determine body fat. As is known in the art, for pregnant subjects, the most useful indicators of obesity are those that do not rely on waist measurements or abdominal height. In one embodiment, total weight, skin-fold test or BMI are used as indicators of obesity for pregnant women.

Calculation of BMI can be readily achieved once the height and weight of the subject are known. Standard equations for calculating BMI are provided above. A subject with a BMI of greater than or equal to 30 is generally accepted to be obese. This value may, however, vary according to characteristics of the individual. For example, for women over 65, a “cut-off” value of 25 has been recommended.

Waist-to-hip ratio and waist circumference can also provide an indication of obesity. Waist-to-hip ratio is calculated by dividing the circumference of the waist by the circumference of the hips. A waist-to-hip ratio of over 1.0 is considered obese for women and men. In general, men with a waist measurement exceeding 40 inches and women with a waist measurement of 35 inches or greater are considered to be obese.

Measurement of body fat can also be used to determine obesity. Body fat can be measured using standard techniques such as the skin-fold test and total body immersion. Skinfold thickness is measured on the trunk and the extremities for assessment of subcutaneous fat patterning. Typically the biceps and triceps skinfolds on the arm, and the suprailiac, subscapular and paraumbilical skinfolds on the trunk are used.

Abdominal height refers to the height that the abdomen rises above the torso when the subject lies on their back. The conicity index, which evaluates waist circumference in relation to height and weight, is calculated according to the equation:
Conicity index=waist circumference (m)/(0.109×square root of weight (kg)/height (m))

In one embodiment of the present invention, the method uses BMI as an indicator of obesity.

Methods of measuring other factors associated with hypertensive status of a subject, such as blood pressure, pulse pressure, cardiac output, aortic stiffness, vascular resistance, and heart rate variability are well known in the art. Systolic or diastolic blood pressure, or a combination of both systolic and diastolic, can be measured. Aortic stiffness and vascular resistance can be measured by standard techniques, such as pulse wave analysis and strain gauge plethysmography. Heart rate variability can be measured, for example, by ECG or the use of intraneural microelectrodes.

Many of these factors can be measured with the subject in standing, sitting or supine positions. As blood pressure in pregnant women is known to be very labile, taking such measurements with the subject in the supine position can help to reduce fluctuations in the measurements for these subjects.

In accordance with one embodiment of the present invention, the method comprises measuring the blood pressure of the subject. In another embodiment, the blood pressure measurement is systolic blood pressure. In a further embodiment, the blood pressure measurement is taken with the subject in a supine position.

Biomarkers

In accordance with the present invention, the method comprises measurement of the level of sFlt-1 in a sample obtained from the subject. The method can also optionally comprise the measurement of one or more other biomarkers known in the art to be associated with the pre-eclampsia. Suitable examples include, but are not limited to, PlGF, soluble endoglin, VEGF, activin and angiotensin II type 1 receptor.

In general, the level of the selected biomarker(s) is measured is a sample of bodily fluid, such as a blood sample (for example, a whole blood, plasma or serum sample), urine, saliva, amniotic fluid, or cerebrospinal fluid. However, one skilled in the art will appreciate that other types of samples are also appropriate, for example, a tissue sample such as a placental tissue sample. In one embodiment of the present invention, the sample is a blood, urine or amniotic fluid sample. In another embodiment, the sample is a serum sample. The sample can be used either directly as obtained from the subject or following a pre-treatment to modify the character of the sample. Thus, the biological sample can be pre-treated prior to use by, for example, preparing plasma or serum from blood, disrupting cells, preparing liquids from solid materials, diluting viscous fluids, filtering liquids, distilling liquids, concentrating liquids, inactivating interfering components, adding reagents, and the like.

Methods of measuring the levels of biomarker(s) are known in the art and generally comprise measuring the level of the protein, using for example, a monoclonal antibody or other appropriate binding partner, or the level of the mRNA encoding the protein, using for example, appropriate polynucleotide primers and/or probes. Appropriate binding partners include natural and synthetic ligands, receptors, fragments of receptors or other molecules that bind to the protein of interest with sufficient strength and specificity. Measurement of autoantibodies can also be used to determine the levels of serum angiotensin II type 1 receptor (see, for example, Dechend R, et al. (2003) Circulation 107:1632-1639; Xia, et al. (2003) J Soc Gynecol Investig 10:82-93).

Methods for detecting proteins include, for example, ELISAs, Western blotting, immunoassays, including sandwich assays, reverse sandwich assays and modified sandwich assays, and the like, as generally described in Coligan et al. (Current Protocols in Immunology, Wiley Interscience, New York, 2001) and Coligan et al. (Current Protocols in Protein Science, Wiley Interscience, New York, 2001). Methods for detecting nucleic acids, such as mRNA, for example, comprising Northern blot analysis, PCR, RT-PCR, hybridization analysis (for example, using molecular beacon or TaqMan probes), and the like, are known in the art (for example, see Ausubel et al. Current Protocols in Molecular Biology, Wiley Interscience, New York, 2001).

For example, the level of sFlt-1, PlGF, endoglin and VEGF can be measured by ELISA using commercially available kits (R&D Systems, Minneapolis, Minn.). Levels of free (i.e. unbound) sFlt-1, bound sFlt-1 or total sFlt-1 can be measured as is known in the art (see, for example, Belgore, et al. (2001) Clin Sci (Lond) 100(5): 567-75). To measure free sFlt-1, for example, anti-VEGF capture antibodies are first treated with VEGF. The anti-VEGF:VEGF complexes are then used as a capture ligand to bind free sFlt-1 in the sample. sFlt-1 from the sample that binds to the capture ligand can then be detected using a labelled anti-sFlt-1 antibody.

Alternatively, antibodies that bind both bound and free sFlt-1 can be used as capture antibodies and the amount of free sFlt-1 bound to the antibodies subsequently detected using labelled VEGF or PlGF, as described in the Examples provided herein. Suitable samples for use in this assay include serum and plasma samples free of heparin. Samples can be centrifuged prior to use.

In one embodiment, the level of total sFlt-1 in the sample is measured. In another embodiment, the level of free sFlt-1 is measured.

In a further embodiment of the present invention, the method comprises measuring the level of PlGF in the sample. Levels of free (i.e. unbound) PlGF, bound PlGF or total PlGF can be used.

In another embodiment, the method comprises measuring the level of soluble endoglin in the sample. Levels of free endoglin, bound endoglin or total endoglin can be used.

Patients

As noted above, the methods of the present invention can be used to diagnose the hypertensive status of a patient who is either a pregnant woman or a patient (e.g., a cancer patient) undergoing treatment with an anti-angiogenic drug. Optionally the methods can be used to diagnose the hypertensive status of a pregnant woman undergoing treatment with an anti-angiogenic drug.

The method of the present invention can be used to assess the hypertensive status of pregnant women at various stages during their pregnancy. In one embodiment, the patient is at or after 20 weeks of gestation.

PLS is associated with a number of anti-angiogenic drugs currently in use, including, but are not limited to, Bevacizumab (BEV or Avastin®), SU11248 (Sunitinib), ABT-869, BAY 43-9006, Sorafenib, PTK 787 (Vatalanib), AG 013736, and Imatinib (STI-571, Glivec, Gleevec®). Accordingly, patients undergoing treatment (e.g., chemotherapy) with one or more of these drugs can benefit from the method of the present invention. Many other anti-angiogenic drugs or treatment regimens are also currently available or in development and the method of the present invention can also be used to determine the hypertensive status of patients undergoing treatment with these drugs or treatment regimens. Examples include, but are not limited to, ZD6474 (Zactima™), AEE 788, Gefitinib (Iressa™), Erlotinib (Tarceva™), AE-941 (Neovastat™), Vatalanib+FOLFOX-4, Vatalanib+FOLFIRI, Somaxanib, Somaxanib+cisplatin/gemcitabine, SU 6668, ADZ 2171, AEE788, Docetaxel+ZD6474, and AG-013736. Anti-angiogenic drugs are used to treat cancer as well as a variety of other diseases, including but not limited to diabetic retinopathy, psoriasis, and rheumatoid arthritis.

In one embodiment, the patient is undergoing or about to undergo treatment with an anti-angiogenic drug that is a receptor tyrosine kinase inhibitor. In another embodiment, the patient is undergoing, or about to undergo, treatment with the anti-angiogenic drug Bevacizumab (BEV or Avastin®), SU11248 (Sunitinib), ABT-869, BAY 43-9006, Sorafenib, PTK 787 (Vatalanib), AG 013736, or Imatinib (STI-571, Glivec®, Gleevec®).

In still another embodiment, the methods of the invention can be employed to assess the effectiveness of treatment of a hypertensive patient, e.g., of a pregnant woman or of a patient undergoing treatment with an anti-angiogenic drug.

Pre-Determined Values

The method of the present invention includes comparing the level or amount of the factor determined for the subject with a pre-determined value. The pre-determined value for a given factor is determined by standard analysis techniques using data comprising measurements of the level or amount of the same factor in a reference population. The pre-determined value can take a variety of forms, for example, it can be single cut-off value, or it can take the form of a range, such as where the reference population is divided equally (or unequally) into groups, for instance, a low-risk group, a medium-risk group and a high-risk group, or into quadrants, the lowest quadrant being individuals with the lowest risk and the highest quadrant being individuals with the highest risk, or into groups or quadrants based on pre-eclampsia severity (i.e. mild, moderate or severe). The pre-determined value can also be established based upon comparative groups, such as where the risk in one defined group is double the risk in another defined group.

The actual numerical value or range of the pre-determined value can vary depending upon the particular reference population selected (for example, size and/or content) and on the assay method employed. For example, a low-risk reference population of women (no family history or other risk factors associated with pre-eclampsia) population may provide a different pre-determined value than will a high-risk reference population, or a population including both low- and high-risk individuals. Accordingly, the pre-determined values selected may take into account the “category” into which an individual falls where this is desired. Pre-determined values for each of the factors included in the method of the invention can be readily determined by standard analytical techniques, and appropriate reference populations can be selected, with no more than routine experimentation by those of ordinary skill in the art.

In one embodiment of the present invention, pre-determined values for the factors included in the method are determined by multi-variate analysis.

Multivariate Analysis Method

The present invention further provides for a multivariate analysis method for identifying factors useful for the prediction or diagnosis of the hypertensive status of pregnant women and/or patients undergoing anti-angiogenic treatment (e.g., chemotherapy). The multivariate analysis method can also be employed to generate a functional representation, such as a nomogram or decision tree, of the correlation between various factors that allows the diagnosis of pre-eclampsia.

The multivariate analysis method generally comprises applying multivariate statistical analysis to a combination of factors determined for each of a plurality of subjects having a hypertensive state (the “reference population”) in order to determine those factors that are relevant to the assessment of hypertensive status and to determine appropriate pre-determined (cut-off′) values for each relevant factor. In one embodiment of the present invention, the combination of factors used in the multivariate analysis method comprises at least one biomarker and at least one physical parameter. The multivariate analysis method can also be employed to further refine the above-described diagnostic method for determining hypertensive status by identifying additional factors that allow the diagnosis to be refined, for example, to identify the optimal combination of factors to distinguish pre-eclampsia from other hypertension states, and to update the method as new data becomes available.

Suitable reference populations can be readily selected by one skilled in art based on the intended application of the results of the analysis. For example, if the analysis is to provide a means for determining the hypertensive status of a pregnant woman, then a suitable reference population would be a plurality of women who had been monitored throughout and subsequent to their pregnancy for various hypertension states and for whom appropriate measurements of the factors of interest were available. By way of example, a suitable reference population would include normal subjects and subjects having one of the hypertension disorders of pregnancy (i.e. CHTN, pre-eclampsia, gestational hypertension, or superimposed pre-eclampsia). Exemplary inclusion criteria that could be used to assign subjects to one of these groups are as follows (clinical diagnosis of pre-eclampsia being confirmed retrospectively when hypertension and proteinuria resolve within 12-26 weeks after delivery):

Normal Pregnancy: Normotensive (blood pressure (BP)<140/90); No history of cardiovascular disease; No physical or known laboratory evidence of any organ dysfunction; No prescribed medication (except iron, folic acid and/or prenatal vitamins).

Chronic Hypertension: Hypertension (BP ≧140/90 mm Hg) or documented need for antihypertensive medications before pregnancy, or noted before the 12th week of gestation; Hypertension diagnosed for the first time during pregnancy that does not resolve postpartum.

Pre-eclampsia: Gestational blood pressure elevation (BP >140/90 mmHg in a subject known to have been normotensive prior to conception) accompanied by new proteinuria (24 hour urine >300 mg/24 h or urine dipstick 2+); Subject is documented to be normotensive before pregnancy or within 26 weeks after pregnancy; Pre-eclampsia is suspect when hypertension (with or without proteinuria) is accompanied by the signs and symptoms of headache, blurred vision, and abdominal pain, with abnormal laboratory tests, specifically, low platelet counts and abnormal liver enzymes or new hyperuricemia.

Gestational Hypertension: Transient hypertension of pregnancy if pre-eclampsia is not present at the time of delivery and blood pressure returns to normal by 12 weeks postpartum (a retrospective diagnosis).

Similarly, an example of a suitable reference population for an analysis to provide a means for determining the hypertensive status of a subject undergoing anti-angiogenic therapy would be a plurality of patients currently undergoing an anti-angiogenic drug therapy regimen, who had been monitored throughout the regimen and, where applicable, subsequent to termination of the regimen for various hypertension states and for whom appropriate measurements of the factors of interest were available. In one embodiment of the present invention, the subjects included in the reference population for an analysis to provide a means for determining the hypertensive status of a subject undergoing anti-angiogenic therapy, include subjects receiving an anti-angiogenic drug as part of a treatment regimen (e.g., chemotherapy regimen) who have one or more known pre-eclampsia risk factors, as well as subjects receiving an anti-angiogenic drug as part of a treatment regimen (e.g., chemotherapy regimen) who have no known pre-eclampsia risk factors. Examples of pre-eclampsia risk factors include, but are not limited to, race, advanced age (for example >65 yrs), obesity, diabetes mellitus, chronic hypertension, previous pre-eclampsia (if female), family history of pre-eclampsia, Factor V Leiden deficiency and renal disease.

Reference populations can also be selected based on certain desired characteristics or defined criteria, for example, the severity of symptoms associated with a subject's hypertensive status, medical and/or family history, previous pregnancies, stage of pregnancy, prior chemotherapy, cycle of anti-angiogenic therapy, and the like.

Similarly, the present invention contemplates that characteristics other than the biomarkers and physical parameters may be included in the multivariate analysis method. Such characteristics include other known risk factors, patient age, gender (where appropriate), ethnicity, socioeconomic background, previous therapies, and the like, and may be used to refine or “tailor” the results for a specific application. For example, the multivariate analysis method may include one or more of the known risk factors for pre-eclampsia during pregnancy as outlined in Table 1.

TABLE 1
Risk Factors for Pre-Eclampsia
Risk Factors¹

| | |
|---|---|
| Nulliparity | Chronic Hypertension |
| Hydatidiform mole | Intracytoplasmic Sperm Injection |
| | (ICSI), Donor Insemination, |
| | Oocyte Donation, Embryo Donation |
| Hydrops fetalis | Black Race |
| Family History of Pre-eclampsia | Hispanic Race |
| <20 years of age | Pre-pregnancy obesity (e.g. BMI ≧35) |
| Multifetal Gestation | Pregestational Diabetes Mellitus |
| Previous Pre-eclampsia | Anticardiolipin antibody syndrome or |
| | thrombophilias |
| Polycystic Ovary Disease | Maternal Susceptibility Genes |
| Insulin Resistance | Presence of antiphospholipid antibodies |
| Twin pregnancy | >35 years of age (especially age ≧40) |
| ≧10 years between pregnancies | Chronic autoimmune disease |
| Renal Disease | Maternal Infections |

¹Source: Duckitt, et al. (2005) BMJ. 330(7491): 565.

In one embodiment of the present invention, at least two of the combination of factors included in the multivariate analysis are levels of sFlt-1 and an indicator of obesity (such as BMI). In another embodiment, at least two of the combination of factors included in the multivariate analysis are levels of sFlt-1 and blood pressure. The multivariate analysis can also be used to identify additional factors that permit the hypertensive status of the subject under investigation to be further refined. For example, in one embodiment of the invention, levels of PlGF are included in the multivariate analysis together with levels of sFlt-1 and an indicator of obesity. In another embodiment, levels of soluble endoglin are included in the multivariate analysis together with levels of sFlt-1 and an obesity factor. Inclusion of additional factors can, for example, allow for the relative risk that a subject will develop PE to be determined at various stages of pregnancy.

In other embodiments, heart rate variability (HRV), strain gauge plethysmography (FVR) and/or pulse wave analysis measurements are included in the multivariate analysis method.

The multivariate method of the present invention can also be employed to correlate a plurality of factors determined for each of a plurality of subjects (the reference population) having a hypertensive state with the occurrence of the hypertensive state in order to generate a functional representation of the correlation, such as a nomogram or decision tree, wherein at least two of the plurality of factors are levels of sFlt-1 and a physical parameter. If desired, the multivariate analysis method can also be used to periodically update the nomogram or decision tree as additional data is obtained. Thus, one embodiment of the invention provides for the use of the multivariate analysis method as part of a “self-learning” system.

In another embodiment, the present invention provides for the use of the multivariate analysis method to develop a nomogram or decision tree to diagnose a subject's hypertensive status during the administration of anti-angiogenic drugs (for example to distinguish CHTN from pre-eclampsia).

Standard multivariate analysis techniques known in the art can be used (see, for example, Armitage, et al. ((2002). Multivariate methods. In: Statistical Methods in Medical Research, Blackwell Science. Malden, Mass. pp 455-484; Breiman, L., Friedman, J. H., Olshen, R. A. and Stone., C. J. (1983). “Classification and Regression Trees.” Wadsworth).

In one embodiment of the present invention, the multivariate analysis method comprises analysis of the factors by principal component analysis (PCA). Principal components analysis (PCA) is a multivariate statistics technique for simplifying a dataset. It is a linear transformation that transforms the data to a new coordinate system such that the greatest variance by any projection of the data comes to lie on the first coordinate (or first principal component), the second greatest variance on the second coordinate (second principal component), and so on. PCA can be used for dimensionality reduction in a dataset while retaining those characteristics of the dataset that contribute most to its variance, by keeping lower-order principal components and ignoring higher-order ones. Such low-order components often contain the more important aspects of the data, although depending on the application this may not always be the case.

In another embodiment of the present invention, the multivariate analysis method further comprises discriminant function analysis (DFA) and/or neural network analysis.

Apparatus

The present invention further provides for an apparatus for diagnosing a subject's hypertensive status. The apparatus comprises a correlation of a plurality of factors determined for each of a plurality of subjects having a hypertensive state with the occurrence of the hypertensive state in each of the subjects. Suitable factors are described above. The correlation can be, for example, in the form of a nomogram. The apparatus further includes a means for (i.e., is configured to permit) matching an identical set of factors determined for a subject of interest to the correlation to diagnose the hypertensive status of the subject.

In one embodiment, the plurality of factors comprises two or more factors. In another embodiment, the plurality of factors comprises three or more factors. In a further embodiment, at least two of the plurality of factors are levels of sFlt-1 and an indicator of obesity (such as BMI).

The apparatus can take one of a variety of forms, for example, the correlation and means of matching can be provided as a computer program, for example in Palm (including Treo 600), Pocket PC, or Flash 6.0 format, in which case, the apparatus can be a computer software product, a hand-held device, such as a Palm Pilot or Blackberry, or it can be a world-wide-web (WWW) page, or it can be a computing device. Alternatively, the apparatus can be a simple functional representation of the correlation such as a nomogram provided on a card, or wheel, that is readily portable and simple to use. For example, the apparatus can be in the form of a laminated card or wheel. Accordingly, the correlation can be a graphic representation, which, in some embodiments, is stored in a database or memory, such as a random access memory, read-only memory, disk, virtual memory or processor. Other suitable representations, pictures, depictions or exemplifications known in the art may also be used.

The apparatus may further comprise a storage means for storing the correlation or nomogram, an input means that allows the input into the apparatus of the identical set of factors determined for a subject, and a display means for displaying the hypertensive status of the subject. The storage means can be, for example, random access memory, read-only memory, a disk, virtual memory, a database, or a processor. The input means can be, for example, a keypad, a keyboard, stored data, a touch screen, a voice-activated system, a downloadable program, downloadable data, a digital interface, a hand-held device, or an infrared signal device. The display means can be, for example, a computer monitor, a cathode ray tub (CRT), a digital screen, a light-emitting diode (LED), a liquid crystal display (LCD), an X-ray, a compressed digitized image, a video image, or a hand-held device. The apparatus can further comprise a database, wherein the database stores the correlation of factors and is accessible to the user.

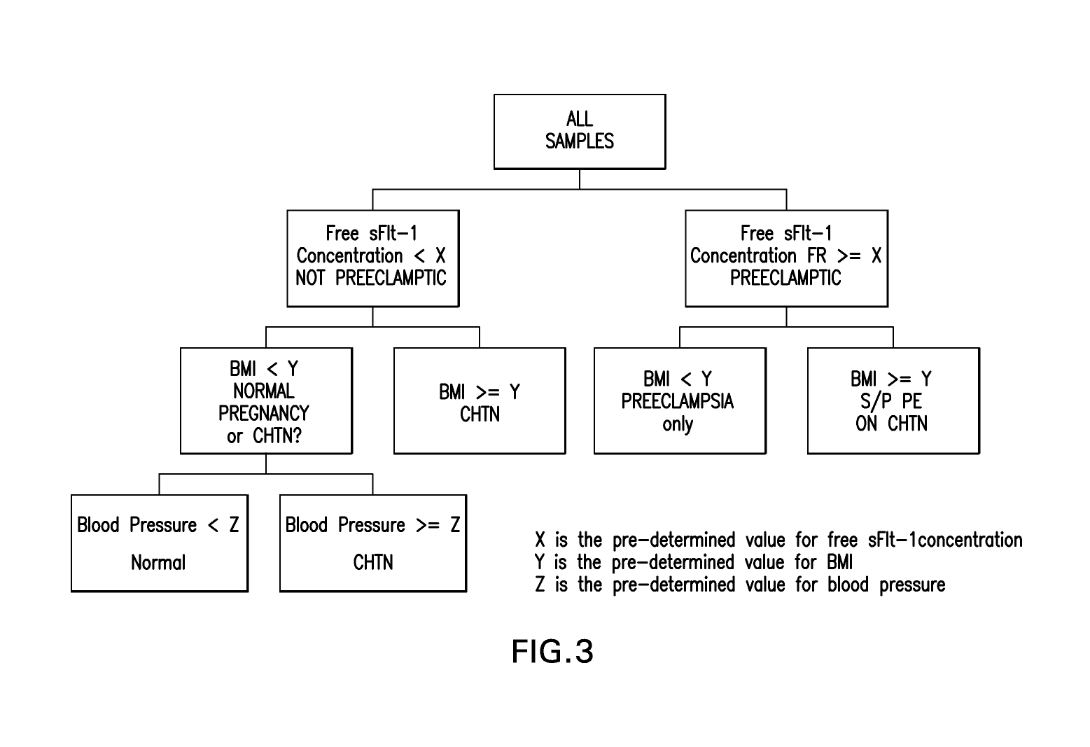

One embodiment of the present invention provides for an apparatus in simple manual format comprising a solid support having disposed thereon a graphic representation of the correlation, for example, in the form of a nomogram. The graphic representation can comprise, for example, nomogram indicia means comprising a scale for each of the plurality of factors and a diagnosis scale. For example, the nomogram indicia means can comprise a sFlt-1 level scale, an obesity factor scale and a diagnosis scale. The factor scales are disposed on the solid surface such that the values on these scales can be correlated with the diagnosis scale in order to diagnose a subject's hypertensive state. For example, the scales can take the form of lines disposed on the solid surface such that by aligning a straight edge with the known values on the factors scales, a diagnosis can be read off the diagnosis scale. Alternatively, the apparatus can take the form of a plurality of superimposed circular solid surfaces such that the known values for each factor can be “dialed in” on the appropriate factor scale and the diagnosis read off the diagnosis scale. Another alternative contemplated by the present invention is a graphical representation in the form of a decision tree, such as the embodiment shown in FIG. 3. Other suitable representations, pictures and depictions are known in the art.

In another embodiment of the present invention, the apparatus is a computer program product such as a solid or fluid transmission medium, magnetic or optical wire, tape or disc, memory stick, or the like, for storing signals readable by a machine.

In another embodiment of the present invention, the apparatus is a computing device, for example, in the form of a computer or hand-held device that includes a processing unit, memory, and storage. The computing device can include, or have access to a computing environment that comprises a variety of computer-readable media, such as volatile memory and non-volatile memory, removable storage and/or non-removable storage. Computer storage includes, for example, RAM, ROM, EPROM & EEPROM, flash memory or other memory technologies, CD ROM, Digital Versatile Disks (DVD) or other optical disk storage, magnetic cassettes, magnetic tape, magnetic disk storage or other magnetic storage devices, or other medium known in the art to be capable of storing computer-readable instructions. The computing device can also include or have access to a computing environment that comprises input, output, and/or a communication connection. The input can be one or several devices, such as a keyboard, mouse, touch screen, or stylus. The output can also be one or several devices, such as a video display, a printer, an audio output device, a touch stimulation output device, or a screen reading output device. If desired, the computing device can be configured to operate in a networked environment using a communication connection to connect to one or more remote computers. The communication connection can be, for example, a Local Area Network (LAN), a Wide Area Network (WAN) or other networks and can operate over a wired network, wireless radio frequency network, and/or an infrared network.

Optionally the apparatus can be part of or have remote access to the means for carrying out the measure of levels of biomarker(s).

Application of Method and Apparatus

The method and apparatus of the present invention can be used, for example, by a physician to diagnose the hypertensive status of a patient in his or her care, or by a patient for self-diagnosis. Knowing a patient's hypertensive status can facilitate decisions with respect to an appropriate treatment regimen or course of action for the patient.

For example, with respect to pregnant women, accurate diagnosis of the woman's hypertensive status, and in particular differential diagnosis between pre-eclampsia, CHTN and pre-eclampsia superimposed on CHTN, can help guide decisions with respect to administration of anti-hypertensive drugs (timing, type of drug, and the like), appropriate bed-rest, pre-term delivery, peripartum administration of magnesium, and other treatment options. For example, the method and apparatus can allow a physician to determine whether anti-hypertensive therapy should be initiated. Commonly used anti-hypertensive drugs are shown in Table 2. Parenteral hydralazine, labetalol and short acting oral nifedipine are the most commonly used drugs for the urgent treatment of severe pre-eclampsia and the American Academy of Pediatrics considers these agents to be compatible with breast feeding [Committee on Drugs, 1994 #19837]

TABLE 2
Commonly Used Antihypertensive Treatments
for Severe Pre-eclampsia

| Medicine | Dose | Notes |
|---|---|---|
| Hydralazine | 5 mg IV or IM, then 5-10 | NHBEP drug of choice. |
| | mg q 20-40 min | |
| | OR 0.5-10 mg/h infusion | |
| Labetalol | 20 mg IV then 20-80 mg q | Less risk for tachycardia |
| | 20-30 min <300 mg | and arrhythmias. Risk for |
| | OR 1-2 mg · min infusion | neonatal bradycardia |
| Nifedipine | 10-30 mg po q 45 min prn | May effect labor |
| (short acting) | | synergistic with MgSO4 |
| Nitroprusside | 0.5-10 μg · kg/min | Drug of last resort. |
| | | Possible Cyanide toxicity. |
| Methyldopa | 0.5-3 g/day in two divided | NHBEP drug of choice. |
| | doses | |
| Labetolol | 200-1200 mg po QD in 2-3 | Neonatal bradycardia |
| | divided doses | |

Similarly, accurate diagnosis of the hypertensive status of a patient undergoing, or considering, anti-angiogenic drug therapy can facilitate decisions as to whether anti-angiogenic drug therapy should be initiated, whether an anti-angiogenic drug regimen in progress should be modified or stopped, whether additional drugs such as anti-hypertensives should be added to the regimen, and the like.

In addition to assisting the physician and/or patient in selecting an appropriate course of therapy, the method and apparatus of the present invention are also useful in the design of clinical trials to evaluate therapies for treating or managing various hypertensive states, including pre-eclampsia associated with pregnancy and pre-eclampsia associated with anti-angiogenic drug therapy. For example, to identify patients appropriate for inclusion in a trial, to verify the effectiveness of randomization, to reduce the sample size requirements, and to facilitate comparisons across studies. Similarly, the method and apparatus can be used to assess or monitor patients during trials relating to the development of anti-angiogenic agents.

Treatment guidelines with respect to peripartum antihypertensive therapy generally reflect results of meta-analyses and the consensus of experts, rather than clinical trials in which hypertensive gravidas, with specific diagnosis of the disorders leading to their hypertension, are randomized to differing levels of blood pressure control. Thus the method of the present invention, which allows varying HDP to be distinguished, will be useful to select, categorize and/or randomize patients for inclusion in such clinical trials.

The invention will now be described with reference to specific examples. It will be understood that the following examples are intended to describe embodiments of the invention and are not intended to limit the invention in any way.

### EXAMPLES

### Example 1

### Development of a Decision Tree to Distinguish Pre-Eclampsia, CHTN and Pre-Eclampsia Superimposed on CHTN in Pregnant Women

Study Design: This was a prospective, multicenter (Washington Hospital Center (WHC) & Georgetown University (GUH)), non-randomized, non-blinded, open-label, diagnostic (non-interventional) pilot-study consisting of normotensive and hypertensive pregnant women aged 18-50 yrs of gestational age 20-40 wks. Subjects were recruited by their physician, either at their clinic office, or at labor and delivery suites, located at WHC and GUH. Inclusion criteria are based on NHLBI Working Group on Research on Hypertension During Pregnancy diagnostic guidelines (as outlined above).

Biosignals: Noninvasive pulse wave analysis (PWA, AtCor), Heart Rate Variability (HRV, AtCor) and Strain Gauge Plethysmography (SGP, Hokanson) performed up to weekly and at one session postpartum (A post-partum session was arranged within 26 weeks post-delivery to confirm if pre-eclampsia was the clinical diagnosis).

Subjects: 40 women with complete biosignal and biomarker analysis were investigated and classified into normal, CHTN, PE or PE+CHTN as indicated above in order to discriminate these clinical diagnoses from biosignal and serum biomarker diagnoses. A total of 85 serum samples were taken from the 40 women (all at different gestational weeks). Subject characteristics are summarized in Table 3 below. [Note: This table includes 46 women, including those without serum or urine samples, and further divides them into diabetes mellitus (DM) groups (GDM=Gestational Diabetes Mellitus). Because different subsets of the data were used, there can be a varying numbers of observations. This is due to the fact that for multivariate data analysis, patients (or samples) that had missing observation(s) in any of the variables used for that specific setting of the analysis had to be excluded from that part of the analysis.]

Blood Draw Scheduling and Technique: 5 ml blood sample were taken at initial study, then each week for those women studied prior to delivery and postpartum. Each blood draw consisted of 5 ml whole blood (Red-Top Tube, stored on ice <3 hours) spun down to achieve 2 ml of serum which was frozen (−80° C.<2 yr.) and then thawed prior to use. Urine samples were also taken at the same intervals.

TABLE 3
Subject Characteristics

| | | Race | BMI | | MAP |
|---|---|---|---|---|---|
| Group | | (W + B + | kg/m² | BP | mmHg |
| (Protein) | n | Other) | (Average) | mmHg | (Average) |
| Normal | 8 | 4 + 3 + 1 | 25-43 | 96-128 | 73-93 |
| (0) | | | (30) | 62-72 | (83) |
| GDM | 3 | 1 + 1 + 1 | 43-57 | 130-138 | 83-95 |
| (0-trace) | | | (48) | 56-77 | (87) |
| CHTN | 16 | 5 + 8 + 3 | 27-50 | 115-172 | 79-127 |
| (0-trace) | | | (38) | 60-104 | (98) |
| PE | 17 | 5 + 9 + 3 | 25-51 | 122-192 | 92-127 |
| (>300 mg/d) | | | (31) | 73-100 | (106) |
| PE + CHTN | 5 | 1 + 3 + 1 | 22-57 | 132-180 | 97-111 |
| (>300 mg/d) | | | (36) | 80-95 | (108) |
| PE + CHTN + DM | 3 | 1 + 2 + 0 | 31-38 | 150-162 | 103-114 |
| (>300 mg/d) | | | (35) | 103-114 | (109) |

Methods:

Biosignal analysis: Blood pressure (systolic and diastolic) was measured by standard protocols. Body mass index was calculated according to the equation:

$$BMI = \frac{Weight ( kg )}{Height squared ( {m}^{2} )}$$

Sequential applanation tonometry was used to calculate pulse wave velocity (PWV) between the radial and carotid artery sites, as well as augmentation index (AIx) using the carotid waveform (Cortez-Cooper et al. (2003) Am J Cardiol 91(12): 1519-22, A9), and pulse pressure. PWV is calculated by dividing the distance between carotid and radial sites by the change in pulse wave transfer time (DPWTT) (see Chiu et al. (1991) American Heart Journal 121(5): 1460-9). Augmentation index (AIx) is the ratio of augmented carotid systolic pressure (due to the late systolic peak in the central pressure waveform) to pulse pressure, and represents a measure of a combination of factors related to large arterial function. The Millar arterial pulse sensing tonometer measures the peripheral pulse pressure waveform from which central hemodynamic parameters are calculated using a “generalizable transfer function” algorithm (see Tsai, et al. (2001) Heart Lung 30(6): 437-44).

The effect of the baroreflex upon Blood Pressure (sphygmomanometer), PWV and AIx is performed by having the subject change positions detailed as follows. The subject initially lies comfortably in the left lateral decubitus position, to prevent aortocaval compression, for 10 minutes to allow hemodynamic equilibration. During this time, subject-specific information is entered into the computer's database and surface measurements of the distance (mm) from the sternal notch to the pulse location of the radial and carotid arteries are made. The tonometer is placed over each palpable pulse to detect the arterial waveform. Pulse wave velocity is determined by repeated tonometric measures while gated ECG measurements are made from three peripheral limb leads (±2 chest leads). The subject is assisted to sit upright while her legs hang over the side of the bed. After a 5 minute wait to re-establish equilibration, all measures are repeated.

Strain Gauge Plethysmography (SGP): Microvascular function was assessed by forearm venous blood flow protocol using Hokanson Silastic strain gauges with the Hokanson Plethysmograph EC5R (see Gamble, et al. (1993) J Physiol 464: 407-422). SGP utilizes strain gauges which indirectly measures changes in blood volume by measuring the circumference of a limb as a cuff is rapidly inflated and deflated. Increased resistance reduces voltage across the gauge which is calibrated to reflect volume changes via a computer display or strip chart recorder (also called volume pulse recording). Extrinsic or vena caval compression due a gravid uterus could possibly affect SPG sensitivity, therefore SGG measures are obtained with the subject in the left lateral decubitus position (see Rumwell, et al. (2000). Part III: Venous Evaluation. Vascular Technology: An Illustrated Review. Pasadena, Calif., Davies Publishing: 169-214). The strain gauge (Hokanson Inc.) is attached to the forearm, connected to Hokanson Plethysmograph, and supported above the level of the right atrium. A cuff is inflated to a pressure of 50 mm Hg above the systolic blood pressure to exclude limb circulation during the measurement of blood flow. The limb's congesting cuff was inflated to 40 mm Hg for 7 seconds in each 15-second cycle to occlude venous outflow from the limb with a period cuff inflator (by hand or with a Hokanson EC-20 cuff inflator). The blood flow output signal is transmitted via the signal processor to the printer or computer and is expressed as ml/min/100 ml of limb volume (see Fehling, et al. (1999) Int J Sports Med 20(8): 555-.).

Biomarker analysis: to assay free sFlt-1 assay, a serum sample was introduced into a reaction vessel followed by the addition of paramagnetic particles to which a monoclonal antibody against sFlt-1 had been attached, which allows capture of the sFlt-1 in the sample with and without bound PlGF. After 18 minutes the sample was removed and the particles washed being held to the side of the reaction vessel by the application of an external magnet. PlGF conjugated to acridinium was then added and allowed to incubate five minutes. The acridinylated PlGF only binds sFlt-1 molecules that have unoccupied binding sites. Excess conjugate was then washed away while being held by a magnet external to the reaction vessel. Chemiluminescence was measured after adding a solution of acid and hydrogen peroxide followed by base to trigger the release of photons. Photomultiplier tubes were used to measure the released photons. The assay was calibrated using various concentrations of recombinant sFlt-1. Results are in pMol/L.

Similarly, to assay PlGF, a sample with PlGF was introduced into a reaction vessel followed by the addition of paramagnetic particles coated with a monoclonal antibody against PlGF that is specific for free PlGF and, therefore, only captures PlGF in the sample that is not complexed with sFlt-1. After 18 minutes the sample was removed and the particles washed while being held to the side of the reaction vessel by the application of an external magnet. Polyclonal goat anti-PlGF antibody conjugated to acridinium was then added and allowed to incubate five minutes. Excess conjugate was then washed away while a magnet holds the particles external to the reaction vessel. Chemiluminescence was measured as described above. The assay is calibrated using recombinant PlGF of various concentrations.

Statistical Analysis: Descriptive and graphical methods were applied as generally described in Armitage, et al. ((2002). Multivariate methods. In: Statistical Methods in Medical Research, Blackwell Science. Malden, Mass. pp 455-484). Dispersion plots and principal components analysis were used to summarize groups of variables.

Results

Initial Clinical vs. Biomarker Diagnosis

The diagnosis of the patients by clinical and biomarker analysis was compared based on the following diagnostic criteria.

Clinical Diagnosis: Pre-eclampsia (PE)=if hypertension and proteinuria resolve within 12-26 weeks postpartum. CHTN=Preexisting hypertension before 20 weeks gestation. (Continued hypertension±proteinuria suggests undiagnosed preexisting CHTN or renal disease, requiring further workup). PE+CHTN=Elevated blood pressure and proteinuria resolved to chronic hypertensive levels within 26 weeks postpartum.

Serum Biomarker Diagnosis: PE=Ratio of normalized serum free sFlt-1 (FR)/PlGF>1, (i.e. FR>2, PlGF<2). CHTN=preexisting hypertension before 20 weeks gestation and FR/PlGF ratio <1. PE+CHTN=preexisting hypertension before 20 weeks gestation and FR/PlGF>1.

The results are shown in Table 4 and indicate that the diagnosis differs depending on whether clinical or biomarker criteria are used.

TABLE 4
Clinical Diagnosis vs. Serum Biomarker
Diagnosis (number of samples)

| Diagnosis | Clinical | Serum Biomarker |
|---|---|---|
| Normal | 33 | 36 |
| CHTN | 19 | 20 |
| PE | 17 | 16 |
| PE + CHTN | 16 | 15 |

Analysis of Physical Parameters (Biosignals)

Principal components analysis of various biosignals: blood pressure (systolic and diastolic), pulse pressure, augmentation index, body mass index and pulse wave analysis, failed to distinguish pre-eclampsia (PE) from the other hypertensive disorders of pregnancy (HDP). This included measuring positional changes (supine and sitting) for pulse pressure, blood pressure, and pulse wave analysis.

Analysis of Biomarkers

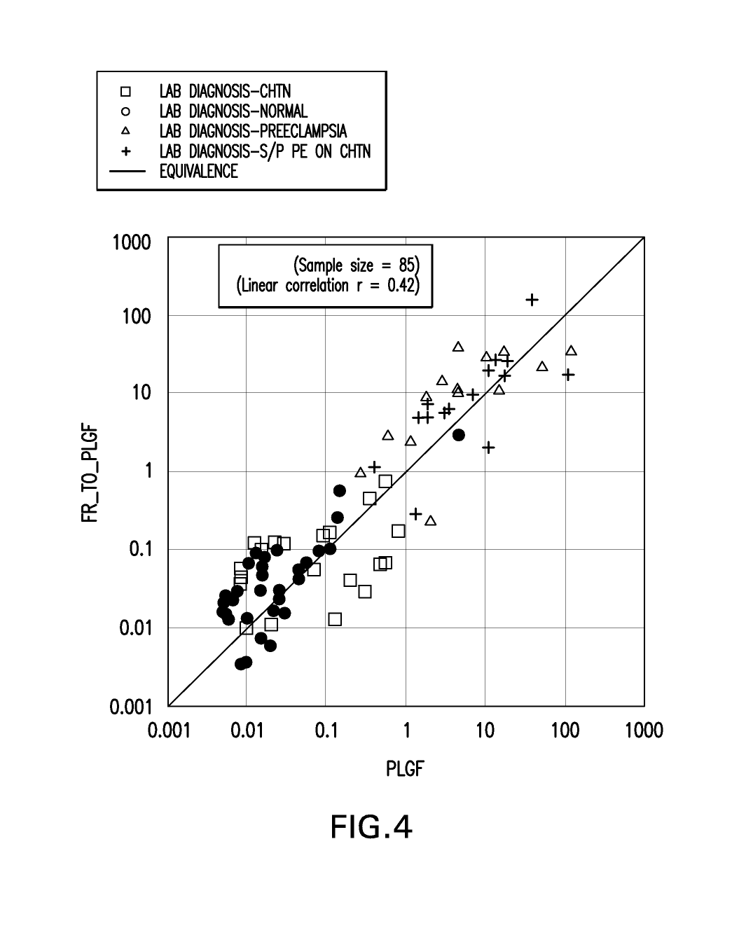

Principal components analysis of sFlt-1 levels and PlGF levels indicated that PE can be distinguished from non-PE (see FIG. 4, which shows a scatter plot of the first principal component (PC) of free sFlt-1 (FR)+PlGF (FR_TO_PlGF) vs. PlGF, MC=Misclassification rate/sample size). If both factor relations were set to a limit of 1, there would be only 2 misclassifications (approximately 2.3%) in terms of correctly assessing PE from non-PE, however, PE+CHTN could not be distinguished from PE.

The results of principal components analysis of free receptor concentration and PlGF are shown in Tables 5 and 6. In Tables 5 and 6, MONO_POLY and FREE_RECEPTOR represent alternative methods of measuring free receptor sFlt-1, and in Table 5 PlGF 1 and PlGF2 represent alternative methods of measuring PlGF.

TABLE 5
Principal Components Analysis of Serum Parameters #1

| Parameter Coefficients | PC1 | PC2 |
|---|---|---|
| MONO_POLY | −0.52319 | 0.295657 |
| FREE_RECEPTOR | −0.48303 | 0.62468 |
| PLGF1 | 0.51989 | 0.26912 |
| PLGF2 | 0.47187 | 0.67077 |
| Variance | 3.345 | 0.508 |
| % of total | 83.626 | 12.690 |
| Cumulative % | 83.626 | 96.316 |

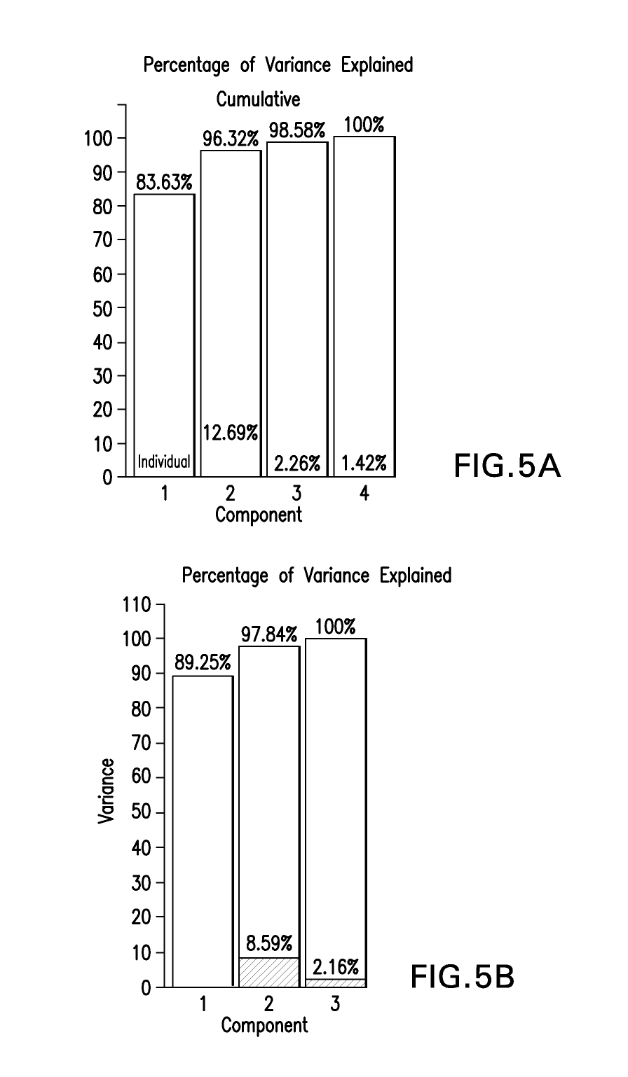

As can be seen from Table 5 and FIG. 5A, the first principal component explains >80% of the variance. The second principal component is no more significant.

TABLE 6
Principal Components Analysis of Serum Parameters #2

| Parameter Coefficients | PCA_F1_SERUM |
|---|---|
| MONO_POLY | −0.5969 |
| PLGF1 | 0.5593 |
| FREE_RECEPTOR | −0.5752 |
| Variance | 2.6774 |
| % of total | 89.25 |
| Cumulative % | 89.25 |

As can be seen from Table 6 and FIG. 5B, the first principal component explains >80% of the variance. The second principal component is no more significant.

Analysis of Physical Parameters and Biomarkers

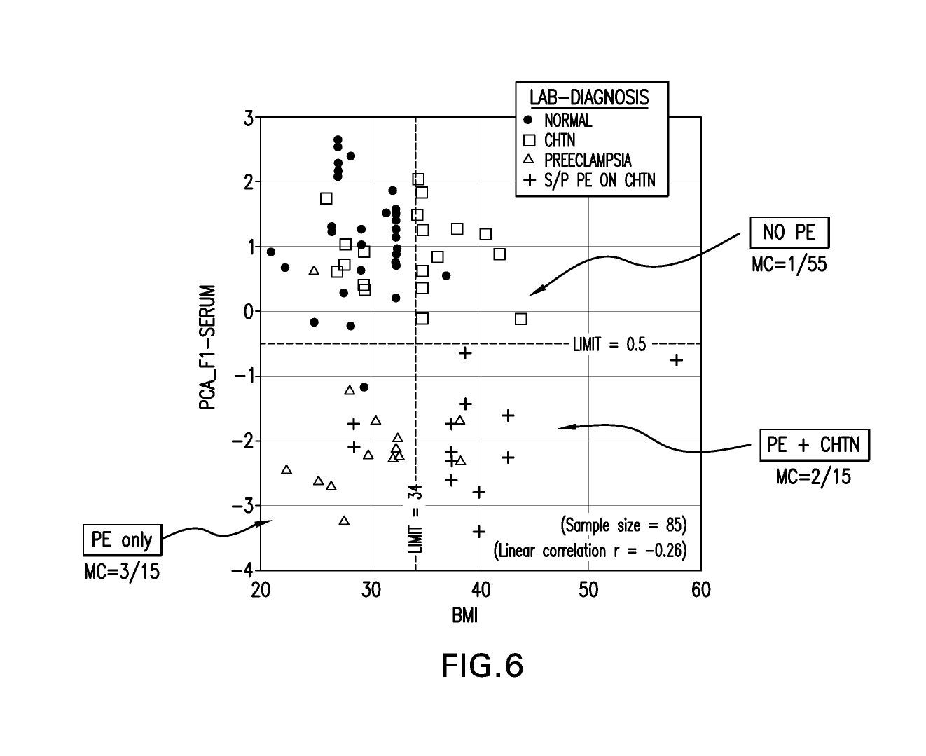

Principal components analysis of free sFlt-1 levels and BMI allowed normotensive and CHTN to be distinguished from PE and PE+CHTN (see FIG. 6, which shows a scatter plot of first PC of free sFlt-1 (PCA_F1 Serum) vs. BMI, MC=Misclassification rate/sample size.) This result clearly indicates that a decision tree or nomogram to distinguish between PE, PE+CHTN and non-PE is possible, which would be simple to use and clinically useful.

Construction of a Decision Tree

Discriminant function analysis (DFA) and neural network analysis was subsequently employed in order to compose a decision tree. The methods and software described in: Terneau TM, Atkinson EJ (1997) “An introduction to recursive partitioning using the RPART routine.” (Technical Report 61, Mayo Clinic, Section of Statistics) were employed. The software is part of the “Insightful Miner™ (available from Insightful Corporation, Seattle, Wash.).

Specifically, the decision tree approach was chosen as an independent method in order to hierarchically split up the total entity of observations into subgroups by importance of factors of influence. Influential variables were determined by searching the top level first for a variable (and cut-off point) to separate into two subgroups via investigation of all factors of influence, and selection of the most influential. This procedure was continued on all subgroups until size of subgroup does not permit any more splits.

A cross-validation step ensures that only the reliable splits were maintained. The minimum number of observations before split was set to n=10, after split to n=5. The splitting criterion was Entropy measure. Stop rule was chosen with a limit on complexity less than 0.001. All potential factors of influence were included on every level, which would allow a repeated subdivision on the same factor multiple times on the different levels (subgroups).

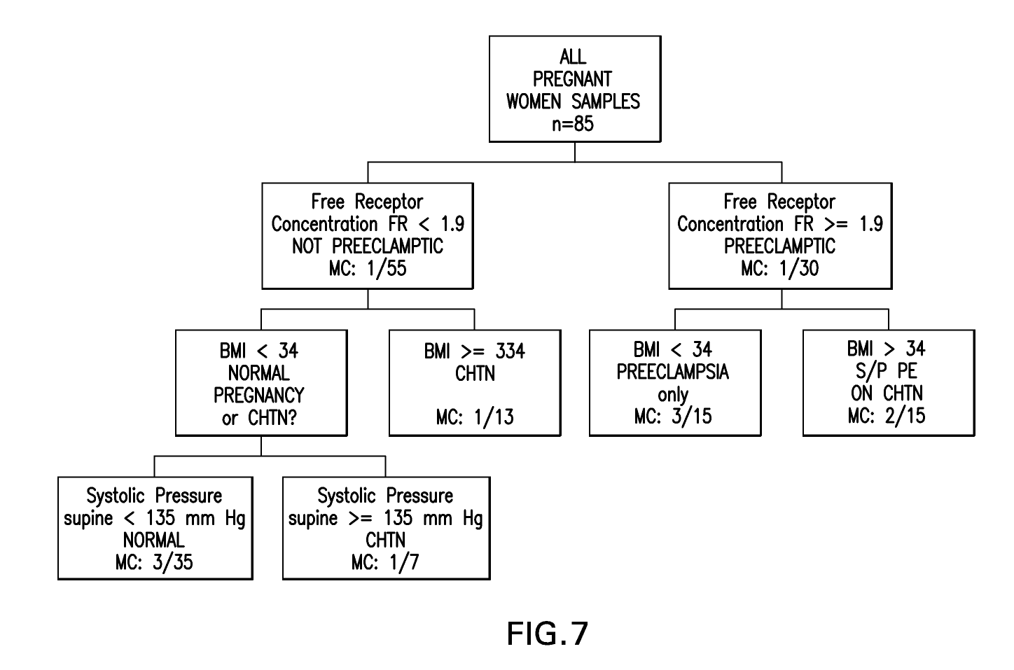

As shown in FIG. 7, three factors: BMI, supine systolic blood pressure and free sFlt-1 serum level were sufficient to compose this decision tree and allow PE, CHTN, normotensive and PE+CHTN to be distinguished. As can also be seen from FIG. 6, two factors: BMI and free sFlt-1 is sufficient to distinguish PE from non-PE, and PE alone from PE+CHTN.

The free sFlt-1 serum level cutoff level for this dataset is 1.9 pMol/L, however, one skilled in the art will appreciate that the sample size used in this analysis was fairly small and that the absolute cutoffs may change slightly as the sample size increases. Similarly, different cut-off values will apply when other assays are used to measure biomarker levels. Appropriate cut-offs can readily be determined by a skilled worker following the methods described above and other standard mathematical techniques.

The disclosure of all patents, publications, including published patent applications, and database entries referenced in this specification are specifically incorporated by reference in their entirety to the same extent as if each such individual patent, publication, and database entry were specifically and individually indicated to be incorporated by reference.

Although the invention has been described with reference to certain specific embodiments, various modifications thereof will be apparent to those skilled in the art without departing from the spirit and scope of the invention as outlined in the claims appended hereto.

We claim:

1. A method for diagnosing the hypertensive status of a subject, said method comprising the steps of:
(a) comparing a measurement of a first factor for said subject to a first pre-determined value, said first factor being a level of sFlt-1 in a sample from said subject, thereby determining the presence or absence of a first hypertensive disorder,
(b) comparing a measurement of second factor for said subject to a second pre-determined value, said second factor being a physical parameter associated with hypertensive status, thereby determining the presence or absence of a second hypertensive disorder, and
(c) diagnosing the hypertensive status of said subject based on the presence or absence of said first and second hypertensive disorders.

2. The method according to claim 1, wherein said physical parameter is an indicator of obesity.

3. The method according to claim 2, wherein said indicator of obesity is weight, body mass index (BMI), waist-to-hip ratio, waist circumference, conicity index, abdominal height, or amount of body fat.

4. The method according to claim 2, wherein said indicator of obesity is body mass index (BMI).

5. The method according to claim 1, wherein said first hypertensive disorder is pre-eclampsia.

6. The method according to claim 5, wherein said second hypertensive disorder is chronic hypertension.

7. The method according to claim 6, wherein said hypertensive status is pre-eclamptic, non-preeclamptic or pre-eclamptic superimposed on chronic hypertension.

8. The method according to claim 1, wherein said level of sFlt-1 is the level of free sFlt-1.

9. The method according to claim 1, wherein said subject is a pregnant woman.

10. The method according to claim 1, wherein said subject is a patient undergoing anti-angiogenic drug therapy.

11. The method according to claim 1, wherein said sample is a blood sample.

12. The method according to claim 1, wherein said sample is a plasma or serum sample.

13. The method according to claim 1, wherein said method further comprises comparing a measurement of one or more other physical parameters or biomarkers or a combination thereof, to corresponding pre-determined values, thereby confirming the presence or absence of said first hypertensive disorder or said second hypertensive disorder.

14. The method according to claim 1, wherein said method further comprises comparing a measurement of a third factor to a third pre-determined value, said third factor being blood pressure, thereby confirming the presence or absence of said first hypertensive disorder or said second hypertensive disorder.

15. The method according to claim 14, wherein said third factor is systolic blood pressure.

16. The method according to claim 14, wherein said third factor is supine systolic blood pressure.

17. The method according to claim 14, wherein said first hypertensive disorder is pre-eclampsia, said second hypertensive disorder is chronic hypertension and comparing said measurement of the third factor to the third pre-determined value confirms the presence or absence of chronic hypertension.

18. A method of evaluating whether a subject would benefit from treatment with an anti-hypertensive drug, said method comprising the steps of:
(a) comparing a measurement of a first factor for said subject to a first pre-determined value, said first factor being a level of sFlt-1 in a sample from said subject, thereby determining the presence or absence of a first hypertensive disorder,
(b) comparing a measurement of second factor for said subject to a second pre-determined value, said second factor being a physical parameter associated with hypertensive status, thereby determining the presence or absence of a second hypertensive disorder, and
(c) diagnosing the hypertensive status of said subject based on the presence or absence of said first and second hypertensive disorders,
wherein the hypertensive status of said subject is indicative of whether said subject would benefit from treatment with an anti-hypertensive drug.

19. The method according to claim 18, wherein said subject is a pregnant woman.

20. The method according to claim 18, wherein said subject is a patient undergoing anti-angiogenic drug therapy.

21. An apparatus for diagnosing the hypertensive status of a subject, said apparatus comprising:
a correlation of a plurality of factors determined for each of a plurality of reference subjects having a hypertensive state with the occurrence of the hypertensive state in each of the reference subjects, said plurality of factors comprising the level of Flt-1 and a physical parameter associated with hypertension, and
a means for matching an identical set of factors determined for said subject to the correlation to diagnose the hypertensive status of the subject.

22. The apparatus of claim 21, wherein said physical parameter is an indicator of obesity.

23. The apparatus of claim 22, wherein said indicator of obesity is weight, body mass index (BMI), waist-to-hip ratio, waist circumference, conicity index, abdominal height, or amount of body fat.

24. The apparatus of claim 22, wherein said indicator of obesity is body mass index (BMI).

25. The apparatus of claim 22, wherein said plurality of factors further comprises blood pressure.

26. The apparatus of claim 21, wherein said apparatus is a computer software product.

27. The apparatus of claim 21, wherein said apparatus comprises a solid support having disposed thereon a graphical representation of said correlation.

28. The apparatus of claim 27, wherein said graphical representation comprises nomogram indicia means, said nomogram indicia means comprising a sFlt-1 level scale, a scale for said physical parameter and a diagnosis scale, wherein the sFlt-1 level scale and the scale for said physical parameter are disposed on said solid surface such that the values on the sFlt-1 scale and the values on the scale for said physical parameter can be correlated with the diagnosis scale to provide a diagnosis of the hypertensive status of said subject.

29. The apparatus of claim 27, wherein said graphical representation comprises a decision tree.

30. A method for identifying factors useful for the diagnosis of the hypertensive status of a subject, said method comprising:
(a) obtaining a data set comprising measurements of a plurality of factors associated with hypertension for each member of a reference population, said reference population comprising subjects each having a hypertensive state of normotensive or having a hypertensive disorder, and
(b) applying multivariate analysis to said data set to correlate said measurements with the hypertensive state of said subjects, thereby identifying factors useful for the diagnosis of the hypertensive status of a subject.

31. The method according to claim 30, wherein said hypertensive disorder is pre-eclamptic, chronically hypertensive, or pre-eclamptic with chronic hypertension.

32. The method according to claim 30, wherein said multivariate analysis comprises principal components analysis.

33. A method of generating a functional representation of a correlation between a plurality of factors associated with hypertension with the hypertensive status of a subject, said method comprising;
(a) obtaining a data set comprising measurements of a plurality of factors associated with hypertension for each member of a reference population, said reference population comprising subjects each having a hypertensive state of normotensive or having a hypertensive disorder;
(b) applying multivariate analysis to said data set to provide a correlation between said measurements and the hypertensive state of said subjects, and
(c) generating a functional representation of said correlation.

34. The method according to claim 33, wherein said hypertensive disorder is pre-eclamptic, chronically hypertensive, or pre-eclamptic with chronic hypertension.

35. The method according to claim 33, wherein said multivariate analysis comprises principal components analysis.

36. The method according to claim 33, wherein said functional correlation is a nomogram or decision tree.

37. A method of diagnosing the hypertensive status of a pregnant subject as pre-eclamptic, non-preeclamptic or pre-eclamptic superimposed on chronic hypertension, said method comprising the steps of:
(a) comparing a measurement of a first factor for said pregnant subject to a first pre-determined value, said first factor being a level of sFlt-1 in a sample from said subject, thereby determining the presence or absence of pre-eclampsia,
(b) comparing a measurement of second factor for said pregnant subject to a second pre-determined value, said second factor being an indicator of obesity, thereby determining the presence or absence of chronic hypertension, and
(c) diagnosing the hypertensive status of said pregnant subject as pre-eclamptic, non-preeclamptic or pre-eclamptic superimposed on chronic hypertension based on the presence or absence of pre-eclampsia and chronic hypertension.

38. The method according to claim 37, wherein said indicator of obesity is body mass index (BMI).

39. The method according to claim 37, wherein said level of sFlt-1 is the level of free sFlt-1.

40. The method according to claim 38, wherein said method further comprises comparing a measurement of a third factor to a third pre-determined value, said third factor being blood pressure, thereby confirming the presence or absence of chronic hypertension.

41. The method according to claim 39, wherein said third factor is systolic blood pressure.

42. The method according to claim 39, wherein said third factor is supine systolic blood pressure.

43. An apparatus for diagnosing the hypertensive status of a subject, said apparatus comprising a correlation of a plurality of factors determined for each of a plurality of reference subjects having a hypertensive state with the occurrence of the hypertensive state in each of the reference subjects, said plurality of factors comprising the level of Flt-1 and a physical parameter associated with hypertension, wherein said apparatus is configured to permit matching an identical set of factors determined for said subject to the correlation to diagnose the hypertensive status of the subject.
